(19) United States
(12) Patent Application Publication
(10) Pub. No.: US 2022/0273841 A1
(43) Pub. Date: Sep. 1, 2022

(54) BONE CEMENT WITH HYALURONIC ACID

(71) Applicants: GRAFTYS, Jumet (BE); UNIVERSITE DE NANTES, Nantes (FR); CENTRE NATIONAL DE LA RECHERCHE SCIENTIFIQUE, Paris (FR)

(72) Inventors: Enrico Javier BASTIANELLI, Rhode-Saint-Genèse (BE); Jean-Michel BOULER, Carquefou (FR); Bruno BUJOLI, Sucé-sur-Erdre (FR)

(73) Assignees: GRAFTYS, Jumet (BE); UNIVERSITE DE NANTES, Nantes (FR); CENTRE NATIONAL DE LA RECHERCHE SCIENTIFIQUE, Paris (FR)

(21) Appl. No.: 17/627,972

(22) PCT Filed: Jul. 17, 2020
(86) PCT No.: PCT/EP2020/070351

§ 371 Date: Jan. 18, 2022

(30) Foreign Application Priority Data
Jul. 19, 2019 (EP) 19187320.7

(51) Int. Cl. A61L 24/00; A61L 27/46

(52) U.S. Cl. CPC A61L 24/0084; A61L 27/46

(57) ABSTRACT

A bone cement composition including a powder component, a liquid component, and hyaluronic acid (HA) or a salt thereof in an amount ranging from 0.01% to 10% w/w in the powder component and/or in the liquid component. The powder component includes calcium phosphate compounds having at least alpha-tricalcium phosphate (α-TCP) and having at least calcium-deficient apatite (CDA). Also, a bone cement obtainable by a process including the following steps: (i) the preparation of a cement composition by mixing the powder component, and the liquid component and (ii) the setting of the cement composition. Further, the use in vitro or ex vivo of a bone cement composition or bone cement in the manufacture of a dental or bony implant.

### FIELD OF INVENTION

The present invention relates to a bone cement composition comprising hyaluronic acid (HA) or a salt thereof, a powder component comprising calcium phosphate compounds and a liquid component. The powder component comprises calcium phosphate compounds comprising at least alpha-tricalcium phosphate (α-TCP) and comprising at least calcium-deficient apatite (CDA). HA or a salt thereof is included in the powder component and/or liquid component in an amount ranging from 0.01% to 10% w/w.

This invention also relates to a bone cement obtainable by setting the bone cement composition according to the invention.

### BACKGROUND OF INVENTION

Bone is a composite of biopolymers consisting mainly of an organic component being collagen and an inorganic component being carbonated hydroxyapatite. Bones may be damaged or necessitate improvement. A wide variety of implant materials have been used to repair, restore and/or augment bone, among which bone cement are very promising. A cement system consists of a powder component and a liquid component, which can be mixed to form a cement paste. The setting of the cement paste lead to the final material (bone cement).

Calcium phosphate cements (CPC) are well-known bone restorative cements, wherein the powder component comprises at least one calcium phosphate compound. Further calcium salts or other additives may be included in small amounts to adjust setting times, increase injectability, increase cohesion or working time and/or introduce macroporosity. The liquid component is usually water-based and may for example be saline, deionized water or an aqueous solution comprising one or more dilute organic or inorganic salts. CPC useful as bone substitutes are disclosed, for example, in EP 1 891 984 A1 and WO 2008/023254 A1 documents (Khairoun, I. et al.).

Bone cements are especially interesting as bone restorative materials due to their malleability, easy handling and precise delivery to various kinds of sites to be treated. Several commercial products are available, including Accufill® (Zimmer), Hydroset® (Stryker), Cerament® (Bone support) and Graftys® HBS and Graftys® QuickSet (Graftys).

Although these products represented significant improvements compared to previously available cements, further improvements are still necessary in order to extend their scope of application and make them suitable for specific clinical indications. Especially, commercially available bone cements may not be sufficiently efficient regarding, for instance, physical properties (e.g., cohesion, viscosity or injectability), setting time, adhesion to bone tissue, blood wash resistance, mechanical properties (e.g., tensile strength, toughness) and/or durability.

Specific additives such as biocompatible and/or bioresorbable polymers (e.g., HPMC or CMC) have been used to adjust the physical and mechanical parameters of cement compositions. However, such additives are as a rule added in the powder component, which can be a limitation depending on the nature of the additive and/or the clinical applications. Especially, the stability of polymers to sterilization methods of the powder component may be too limited (e.g., gamma irradiation can break the polymer chains); the dispersion of the polymer in the powder component may be non-homogeneous ; and/or the dissolution of the polymer during the setting of the cement may be restricted due to insufficient availability of water to solvate the polymer chains.

The Applicant surprisingly found that the addition of hyaluronic acid in a bone cement composition comprising specific calcium phosphate compounds in the powder component significantly improves the physical and mechanical parameters of cement compositions, as well as other clinically relevant properties, and thereby overcome the limitations of prior art bone cement.

### SUMMARY

This invention relates to a bone cement composition comprising:
a powder component comprising:
   calcium phosphate compounds comprising at least alpha tricalcium phosphate (α-TCP) and comprising at least calcium deficient apatite (CDA), and
   optionally at least one calcium sulfate compound;
a liquid component, preferably an aqueous solution or an aqueous suspension; and
hyaluronic acid (HA) or a salt thereof;

wherein
the hyaluronic acid (HA) or salt thereof is comprised in the powder component in an amount ranging from 0.01% to 10% w/w; and/or
the hyaluronic acid (HA) or salt thereof is comprised in the liquid component in an amount ranging from 0.01% to 10% w/w.

According to one embodiment, the powder component comprises the α-TCP in an amount ranging from 60% to 95% w/w; preferably ranging from 70% to 90% w/w; more preferably ranging from 65% to 85% w/w; and the CDA in an amount ranging from 2.5% to 20% w/w; preferably ranging from 5% to 15% w/w.

According to one embodiment, the powder component comprises at least one further calcium phosphate compound selected from hydroxyapatite (HAp), amorphous calcium phosphate (ACP), monocalcium phosphate anhydrous (MCPA), monocalcium phosphate monohydrate (MCPM), dicalcium phosphate dihydrate (DCPD), dicalcium phosphate anhydrous (DCPA), β-tricalcium phosphate (β-TCP), octacalcium phosphate (OCP), tetracalcium phosphate (TTCP) and a mixture thereof; and/or at least one calcium sulfate compound selected from anhydrous calcium sulfate, calcium sulfate hemihydrate, calcium sulfate dihydrate and a mixture thereof; preferably at least one further calcium phosphate compound selected from DCPA, DCPD, MCPM and a mixture thereof. In one embodiment, the powder component consists of α-TCP, CDA and DCPA or consists of α-TCP, CDA, DCPD and MCPM.

According to one embodiment, the liquid component further comprises at least one inorganic salt selected from sodium chloride (NaCl), trisodium phosphate (Na₃PO₄), disodium hydrogen phosphate (Na₂HPO₄), monosodium dihydrogen phosphate (NaH₂PO₄) and a mixture thereof; preferably at least one inorganic salt selected from Na₂HPO₄ and a Na₂HPO_{4/}NaH₂PO₄ mixture; in an amount ranging from 0.01% to 20% w/w; preferably ranging from 0.1% to 1% w/w or from 2 to 8% w/w.

According to one embodiment, the liquid component comprises at least one biological liquid selected from blood, plasma, platelets, platelets concentrate, bone marrow, bone marrow concentrate and mixtures thereof.

According to one embodiment, the liquid component consists of water, HA or salt thereof, optionally Na₂HPO₄, and optionally at least one biological liquid as defined above.

According to one embodiment, the HA or salt thereof has a molecular weight ranging from 1 kDa to 5 000 kDa; preferably from 5 kDa to 5 000 kDa; more preferably from 100 kDa to 5 000 kDa.

According to one embodiment, the powder component comprises the HA or salt thereof in an amount ranging from 0.01% to 5% w/w. In one embodiment, the powder component comprises the HA or salt thereof in an amount ranging from 0.1% to 1% w/w. According to one embodiment, the liquid component comprises the HA or salt thereof in an amount ranging from 0.01% to 5% w/w. In one embodiment, the liquid component comprises the HA or salt thereof in an amount ranging from 0.1% to 1% w/w.

According to one embodiment, the liquid component/powder component (L/S) ratio ranges from 0.3 to 0.7 ml/g; preferably ranges from 0.4 to 0.5 ml/g.

According to one embodiment, the bone cement composition is injectable.

According to one embodiment, the bone cement composition further comprises at least one active ingredient; preferably an active ingredient selected from antibiotics, anti-inflammatory drugs, anti-cancer drugs, anti-osteoporosis drugs, bone anabolic drugs, growth factors, water-soluble radiopaque agents, contrast agents and a mixture thereof.

This invention also relates to a bone cement obtainable by a process comprising the following steps:
(i) the preparation of a cement composition comprising hyaluronic acid (HA) or a salt thereof by mixing: a powder component comprising calcium phosphate compounds comprising at least alpha tricalcium phosphate (α-TCP) and comprising at least calcium deficient apatite (CDA), and optionally at least one calcium sulfate compound; and a liquid component, preferably an aqueous solution or an aqueous suspension; wherein the hyaluronic acid (HA) or salt thereof is comprised in the powder component in an amount ranging from 0.01% to 10% w/w, preferably ranging from 0.01% to 5% w/w, more preferably ranging from 0.1% to 1% w/w; and/or the hyaluronic acid (HA) or salt thereof is comprised in the liquid component in an amount ranging from 0.01% to 10% w/w, preferably ranging from 0.01% to 5% w/w, more preferably ranging from 0.1% to 1% w/w; and
(ii) the setting of said cement composition.

This invention also relates to a bone cement composition according to the invention or a bone cement according to the invention, for use in the treatment of a dental or bony defect or fracture.

This invention also relates to the use in vitro or ex vivo of a bone cement composition according to the invention or of a bone cement according to the invention, in the manufacture of a dental or bony implant. This invention also relates to the use in vitro or ex vivo of a bone cement according to the invention, as a scaffold for tissue engineering. This invention also relates to dental or bony implant consisting of a moulding of a bone cement according to the invention.

This invention also relates to kit-of-part for manufacturing a bone cement composition according to the invention or a bone cement according to the invention, comprising: a first part being a powder component comprising: calcium phosphate compounds comprising at least alpha tricalcium phosphate (α-TCP) and comprising at least calcium deficient apatite (CDA), and optionally at least one calcium sulfate compound; a second part being a liquid component, preferably an aqueous solution or an aqueous suspension; and hyaluronic acid (HA) or a salt thereof; wherein the hyaluronic acid (HA) or salt thereof is comprised in the powder component in an amount ranging from 0.01% to 10% w/w; and/or the hyaluronic acid (HA) or salt thereof is comprised in the liquid component in an amount ranging from 0.01% to 10% w/w.

Definitions

In the present invention, the following terms have the following meanings:
“about” preceding a figure means plus or less 10% of the value of said figure.
“between” and “and” used in combination for defining a range of values means that the higher and lower limit of the range are not included therein, i.e., a value selected within the range cannot be equal to the higher limit or to the lower limit. For example, “between 1 and 5” feature includes neither 1 nor 5 values.
“biocompatible” refers to a material eliciting little or no immune response in a given organism, or is able to integrate with a particular cell type or tissue.
“bioresorbable” refers to a material which can be resorbed naturally, preferably resorbed naturally by cells.
“blood wash resistance” of a paste composition refers to is cohesion under blood flow.
“calcium phosphate cement” (noted “CPC”) refers to a cement wherein the powder component comprises or consists of calcium phosphate compounds. The powder component of a CPC preferably comprises at least 50% w/w, preferably at least 75% w/w, more preferably at least 95% w/w of calcium phosphate compounds.
“cement” refers to the material resulting of the setting of a paste resulting from the mixing of a powder component (solid phase) and a liquid component (liquid phase).
“cohesion time” or “cohesiveness” of a composition refers to the minimum time from which the composition does not disintegrate when immersed in a liquid (e.g., aqueous solution).
“compressive strength” refers to the maximal compressive stress supported by a cement sample upon failure. It is expressed in MPa [Mnewtons/m²].
“fatigue resistance” refers to a desired property of a bone implant, which can break upon multiple deformation (below the max compressive strength value) of the bone or another tissue in contact with the bone implant (e.g., weeks or months after being implanted).
“fragility” refers to an undesired property of a bone implant, which breaks before any deformation by crack propagation
“hyaluronic acid” (noted “HA”) refers to a natural or synthetic polysaccharide having a structure composed of disaccharides units consisting of D-glucuronic acid and N-acetyl-D-glucosamine, linked via alternating β-(1→4) and β-(1→3) glycosidic bonds (CAS n° [9004-61-9], for HA in acid form).
“injectable” refers to a composition which is sufficiently fluid to be extruded with a syringe in a bone cavity without modifying its integrity (e.g. filter pressing). An injectable composition will generally be able to flow through a needle with a diameter of a few millimetres; preferably a diameter ranging from 1 to 5 mm
“implant” refers to an object introduced in the body to replace in part or entirely a tooth, a joint, a bone or a cartilage; preferably a tooth or a bone.

“macropore” refers to a pore with an equivalent diameter higher than 80 μm, preferably ranging from 100 to 300 μm.

“macroporosity” refers to the structure of cement which contains macropores. A “macroporosity higher than 200” means that the macropores of the cement have in average an equivalent diameter higher than 200 μm.

“mesopore” refers to a pore with a diameter ranging from 10 μm to 80 μm. “mesoporosity” refers to the structure of cement which contains mesopores. “microparticle” refers to a particle having a diameter lower than or equal to 1 mm
“micropore” refers to a pore with a diameter lower than 10 μm.

“microporosity” refers to the structure of cement which contains micropores.
“porosity” of a solid material refers to the ratio of the volume of the void in the material to the total volume of the material.
“ranging from” and “to” used in combination for defining a range of values means that the higher and lower limits of the range are included therein, i.e., a value selected within the range may be equal to the higher limit or to the lower limit. For example, “ranging from 1 to 5” feature includes both 1 and 5 values.
“setting” of a cement refers to the hand-off auto-hardening at room or body temperature of the paste resulting from the mixing of the powder component and the liquid component.
“toughness” refers to the ability of a material to absorb energy and plastically deform without fracturing. One definition of material toughness is the amount of energy per unit volume that a material can absorb before rupturing. It may also be defined as a material's resistance to fracture when stressed. Fatigue resistance and fragility of a material are related with its toughness.

### DETAILED DESCRIPTION

Cement composition

This invention relates to a cement composition (also referred to as “cement paste”) comprising:
a powder component comprising calcium phosphate compounds;
a liquid component; and
hyaluronic acid (HA);
wherein
   hyaluronic acid (HA) is comprised in the powder component in an amount ranging from 0.01% to 10% w/w (“w/w” meaning in this context “in weight by weight of the total weight of the powder component”); and/or
   hyaluronic acid (HA) is comprised in the liquid component in an amount ranging from 0.01% to 10% w/w (“w/w” meaning in this context “in weight by weight of the total weight of the liquid component”).

In other words, at least one component selected from the powder component and the liquid component comprises HA; and HA is comprised in each of selected component in an amount ranging from 0.01% to 10% w/w (“w/w” meaning in this context “in weight by weight of the total weight of the selected component”).

According to one embodiment, hyaluronic acid (HA) is comprised in the powder component. In one embodiment, hyaluronic acid (HA) is comprised in the powder component only. According to one embodiment, hyaluronic acid (HA) is comprised in the liquid component. In one embodiment, hyaluronic acid (HA) is comprised in the liquid component only. In one embodiment, hyaluronic acid (HA) is comprised both in the powder component and in the liquid component.

According to one preferred embodiment, the cement composition is a bone cement composition (also referred to as “bone cement paste”). A bone cement may be obtained by setting of the cement composition. According to one embodiment, the bone cement is a calcium phosphate cement (CPC).

According to one preferred embodiment, the powder component comprises calcium phosphate compounds comprising at least alpha-tricalcium phosphate (α-TCP, Ca₃(PO₄)2, CAS n° [7758-87-4]) and comprising at least calcium-deficient apatite (CDA). The Applicant surprisingly found that the use of this specific combination of calcium phosphate compounds with hyaluronic acid leads to especially advantageous properties of the bone cement composition and/or bone cement.

“Calcium-deficient apatite” or “calcium-deficient hydroxyapatite” or “CDA” are synonyms and refer to a compound of general formulae Ca₁₀₋ₓ[ ]ₓ(HPO₄)_{y}(PO₄)_{6-y}(OH)_{2-z}[ ]z, wherein x, y and z are integers.

According to one embodiment, the powder component comprises the α-TCP in an amount ranging from 50% to 99% w/w; preferably ranging from 60% to 95% w/w; more preferably ranging from 70% to 90% w/w; furthermore preferably ranging from 65% to 85% w/w (“w/w” meaning in this context “in weight by weight of the total weight of the powder component”).

According to one embodiment, the powder component comprises the CDA in an amount ranging from 1% to 25% w/w; preferably ranging from 2.5% to 20% w/w; more preferably ranging from 5% to 15% w/w (“w/w” meaning in this context “in weight by weight of the total weight of the powder component”).

According to one embodiment, the powder component comprises at least one calcium sulfate compound. According to one embodiment, the calcium sulfate compound is selected from anhydrous calcium sulfate (or “anhydrite”, CaSO₄), calcium sulfate hemihydrate (or “bassanite”, CaSO₄′0.5H₂O), calcium sulphate dihydrate (or “gypsum”, CaSO₄′2H₂O) and a mixture thereof. In one embodiment, the powder component comprises the calcium sulfate compound in an amount ranging from 1% to 75% w/w;

preferably ranging from 2.5% to 30% w/w; (“w/w” meaning in this context “in weight by weight of the total weight of the powder component”).

According to one embodiment, the powder component comprises at least one further calcium phosphate compound selected from hydroxyapatite (HAₚ), amorphous calcium phosphate (ACP), monocalcium phosphate anhydrous (MCPA), monocalcium phosphate monohydrate (MCPM, Ca(H₂PO₄)₂H), dicalcium phosphate dihydrate (DCPD, CaHPO₄·(H₂O)₂), dicalcium phosphate anhydrous (DCPA, CaHPO₄), β-tricalcium phosphate (β-TCP, Ca₃(PO₄)₂, CAS n° [7758-87-4]), octacalcium phosphate (OCP), tetracalcium phosphate (TTCP) and a mixture thereof. In one embodiment, the powder component comprises each of the further calcium phosphate (i.e., each calcium phosphate compounds other than α-TCP and CDA) in an amount ranging from 1% to 25% w/w; preferably ranging from 2.5% to 20% w/w; more preferably ranging from 5% to 15% w/w (“w/w” meaning in this context “in weight by weight of the total weight of the powder component”),In one embodiment, the powder component comprises at least one further calcium phosphate compound selected from DCPA, DCPD, MCPM and a mixture thereof. In one embodiment, the powder component consists of α-TCP, CDA and DCPA or consists of α-TCP, CDA, DCPD and MCPM.

In one embodiment, the powder component has a main population of particle size (i.e., more than 50% of particles in the powder) ranging from 0.1 to 500 nm, preferably ranging from 0.5 to 250 nm. In one embodiment, the powder component has a main population of particle size ranging from 1 to 50 μm, preferably ranging from 3 to 30 μm. In one embodiment, the powder component has a main population of particle size, ranging from 1 to 150 μm, preferably ranging from 2 to 100 μm.

According to one embodiment, the powder component comprises at least one biocompatible and bioresorbable polysaccharide polymer. In one embodiment, the polysaccharide polymer is selected from cellulose ethers, salts thereof and mixtures thereof. In one specific embodiment, the polysaccharide polymer is selected from hydroxypropylmethylcellulose (HPMC) and carboxymethylcellulose (CMC). Biocompatible and bioresorbable polymers can be used as fine powders, fibres or microparticles. The biocompatible and bioresorbable polysaccharide polymer swell in contact with the liquid component and is integrated in the inorganic part of the bone cement after setting. This confers advantageous biomechanical and rheological properties to the bone cement. Moreover, their further degradation results in channels and mesopores interconnected in the bone cement, which increases fluid diffusion and allow its passive resorption by dissolution through the biological fluids and its active resorption through the colonisation of the macropores by osteoclasts.

According to one preferred embodiment, the liquid component comprises water. In one embodiment, the liquid component is an aqueous solution or an aqueous suspension (colloidal). In one embodiment, the liquid component comprises water in an amount ranging from 80% to 99.99% w/w; preferably ranging from 90% to 99.9% w/w; more preferably ranging from 95% to 99.5% w/w (“w/w” meaning in this context “in weight by weight of the total weight of the liquid component”).

According to one embodiment, the pH of the liquid component ranges from 5 to 10, preferably ranges from 6 to 9, most preferably ranges from 7 to 8.

According to one embodiment, the liquid component further comprises phosphoric acid.

According to one embodiment, the liquid component further comprises at least one inorganic salt selected from trisodium phosphate (Na₃PO₄), disodium hydrogen phosphate (Na₂HPO₄), monosodium dihydrogen phosphate (NaH₂PO₄), dipotassium hydrogen phosphate (K₂HPO₄), monopotassium dihydrogen phosphate (KH₂PO₄), ammonium dihydrogen phosphate (NH₄H₂PO₄), sodium chloride (NaCl), sodium carbonate (Na₂CO₃), sodium bicarbonate (NaHCO₃), sodium alginate, sodium chondroitin sulfate and a mixture thereof. In one embodiment, the inorganic salt is selected from trisodium phosphate (Na₃PO₄), disodium hydrogen phosphate (Na₂HPO₄), monosodium dihydrogen phosphate (NaH₂PO₄), sodium chloride (NaCl) and a mixture thereof. In one specific embodiment, the inorganic salt is selected from Na₂HPO₄ and a Na₂HPO_{4/}NaH₂PO₄ mixture. In one embodiment, the liquid component comprises the inorganic salt in an amount ranging from 0.01% to 20% w/w; preferably ranging from 0.1% to 1% w/w or from 2 to 8% w/w.

According to one embodiment, the liquid component comprises at least one biological liquid selected from blood, plasma, platelets, platelets concentrate, bone marrow, bone marrow concentrate and mixtures thereof. Each biological liquid may be anti-coagulated or not. Each biological liquid may be autologous or allogeneic. In one embodiment, the liquid component comprises at least one biological liquid as listed above and is free of HA (i.e., does not comprises HA).

According to one embodiment, the liquid component consists of water, HA, optionally Na₂HPO₄, and optionally at least one biological liquid as defined hereinabove.

According to one embodiment, the powder component and/or the liquid component is free of citric acid (i.e., does not comprises citric acid). According to one embodiment, the powder component and/or the liquid component is free of acetic acid. In one embodiment, the powder component and/or the liquid component is free of organic acids such as citric acid or acetic acid (i.e., does not comprises any organic acid). According to one embodiment, the powder component and/or the liquid component is free of chitosan. According to one embodiment, the powder component and/or the liquid component is free of glucose. In one embodiment, the powder component and/or the liquid component is free of any sugars. According to one embodiment, the powder component and/or the liquid component does not comprise hydroxypropylmethylcellulose (HPMC) and/or carboxymethylcellulose (CMC). In one embodiment, the powder component and/or the liquid component does not comprise any cellulose ethers, salts thereof or mixtures thereof. In one specific embodiment, the powder component and/or the liquid component does not comprise any polysaccharide polymer. According to one embodiment, the powder component and/or the liquid component does not comprise polylactic acid, polyglycolic acid and/or polycaprolactone. In one embodiment, the powder component and/or the liquid component does not comprise any linear polyester polymer or copolymer.

The number of disaccharide units in the hyaluronic acid polysaccharide (noted “n”) is a positive integer higher than or equal to 10. According to one embodiment, n is at least 100; preferably at least 1 000; more preferably at least 10 000. According to one embodiment, n is 100 000 or less; preferably 50 000 or less; more preferably 25 000 or less.

According to one embodiment, the HA has a molecular weight higher than or equal to 0.1 kDa (kg/mol), preferably higher than or equal to 1 kDa; more preferably higher than or equal to 5 kDa; further more preferably higher than or equal to 100 kDa. According to one embodiment, the HA has a molecular weight lower than or equal to 20 000 kDa, preferably lower than or equal to 10 000 kDa; more preferably lower than or equal to 5 000 kDa. In one embodiment, the HA has a molecular weight ranging from 1 kDa to 5 000 kDa; preferably from 5 kDa to 5 000 kDa; more preferably from 100 kDa to 5 000 kDa.

The hyaluronic acid polysaccharide may be linear or branched. According to a first embodiment, the hyaluronic acid is linear, i.e., it is not branched, so that hyaluronic acid polymeric chains are not linked together except for the glycosidic bonds of the main polymeric chain. According to a second embodiment, the HA is branched, i.e., different polymeric chain in the hyaluronic acid are linked together by means of lateral chains (e.g., through hydroxyl or carboxylic acid functions) so that the HA is a polymeric network.

Branched hyaluronic acid polysaccharide may for example be obtained starting from linear hyaluronic acid polysaccharides submitted to a cross-linking method known in the art.

All references to the hyaluronic acid (HA) in the present disclosure include references to enantiomers, salts, solvates, polymorphs, multi-component complexes and liquid crystals thereof.

According to one preferred embodiment, the HA is in the form of a salt thereof; more preferably a pharmaceutically acceptable salt thereof.

Salts of the HA include the acid addition and base salts thereof.

Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen, phosphate/dihydrogen, phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine and zinc salts. Hemi-salts of acids and bases may also be formed, e.g., hemi-potassium and hemi-sodium salts.

Salts of HA may be prepared by standard synthetic methods, e.g., by reacting a free acid with a suitable organic or inorganic base.

According to one embodiment, the HA salt is selected from the potassium salt of HA, i.e., potassium hyaluronate (CAS n° [31799-91-4]) which is noted “KHA” and sodium salt of HA, i.e., sodium hyaluronate (CAS n° [9067-32-7]) which is noted “NaHA”. In one embodiment, the HA salt is sodium hyaluronate (NaHA).

Hyaluronic acid is commercially available in various molecular weight and purity, as well as in aqueous solutions of various HA concentrations. The HA may be a biosynthetic hyaluronic acid polysaccharide isolated from its native environment, e.g., a living organism. The HA may also be a synthetic HA manufactured by any suitable chemical reaction known in the art, especially synthetic methods known in the field of the chemistry of polysaccharides.

According to one embodiment, the HA has not been modified or substituted by thiol groups. According to one embodiment, the HA has not been modified or substituted by sulfhydryl groups. In one embodiment, the HA does not comprise any sulfur atoms.

According to one embodiment, the HA amount in the powder component ranges from 0.01% to 5% w/w. In one embodiment, the HA amount in the liquid component ranges from 0.1% to 1% w/w.

According to one embodiment, the HA amount in the liquid component ranges from 0.01% to 5% w/w. In one embodiment, the HA amount in the liquid component ranges from 0.1% to 1% w/w.

According to one embodiment, the HA amount in the powder component is between 0.01% and 10% w/w. In one embodiment, the HA amount in the powder component is between 0.01% and 5% w/w. In one specific embodiment, the HA amount in the powder component is between 0.1% and 1% w/w.

According to one embodiment, the HA amount in the liquid component is between 0.01% and 10% w/w. In one embodiment, the HA amount in the liquid component is between 0.01% and 5% w/w. In one specific embodiment, the HA amount in the liquid component is between 0.1% and 1% w/w.

According to one embodiment, the HA amount in the powder component or in the liquid component is not about 0.01% w/w. According to one embodiment, the HA amount in the powder component or in the liquid component is not about 0.1% w/w. According to one embodiment, the HA amount in the powder component or in the liquid component is not about 0.5% w/w. According to one embodiment, the HA amount in the powder component or in the liquid component is not about 0.75% w/w. According to one embodiment, the HA amount in the powder component or in the liquid component is not about 1% w/w. According to one embodiment, the HA amount in the powder component or in the liquid component is not about 1.5% w/w. According to one embodiment, the HA amount in the powder component or in the liquid component is not about 2.5% w/w.

According to one embodiment, the HA amount in the powder component or in the liquid component is not about 3% w/w. According to one embodiment, the HA amount in the powder component or in the liquid component is not about 10% w/w.

According to one embodiment, the liquid component/powder component (L/S) ratio ranges from 0.3 to 0.7 ml/g. In one embodiment, the L/S ratio ranges from 0.3 to 0.6 ml/g.

In one specific embodiment, the L/S ratio ranges from 0.4 to 0.5 ml/g.

According to one embodiment, the cement composition further comprises at least one active ingredient. In one embodiment, the active ingredient is selected from antibiotics, anti-inflammatory drugs, anti-cancer drugs, anti-osteoporosis drugs, bone anabolic drugs, growth factors, water-soluble radiopaque agent, contrast agents such as heavy atom-based nanoparticles (e.g., gold, bismuth, gadolinium, ytterbium) and a mixture thereof.

Advantageously, the cement composition is sufficiently cohesive for use as bone substitute. Cohesion of the cement paste is advantageous regarding the use as bone substitute, because it avoids the disintegration or breakdown of the composition in contact with biological fluids when implanted. The cohesion of a composition may be characterized by a cohesion time, which should be as low as possible. Appropriate cohesion time should be lower than 2 min in water at room temperature; preferably lower than 1 min at room temperature. Methods for measuring a cohesion time are well-known in the art.

Advantageously, the cement composition is injectable. Injectability is useful for all bone void filling applications and necessary for minimally invasive surgery, which reduces the risk and pain and improve recovery of the patient. Especially, injectability is advantageous regarding all intraosseous applications (e.g., augmentation of osteoporotic or necrotic bone, subchondroplasty procedures). Methods for measuring injectability are well-known in the art, although there is currently no standardized method. Such methods are based, for example, on the measure of the amount of composition which has been extruded, the force needed to extrude the composition and/or the injection pressure.

According to one embodiment, the cement composition has an applied force for injection (Inj. force) ranging from about 1 to about 20 N; when measured by the following extrusion method: After mixing the solid and liquid components, the cement composition (3.5 mL) was introduced in a syringe (Terumo®) with a 1.5 mm diameter needle. After 2 or 15 min, the paste was extruded by applying a syringe plunger displacement at a speed of 1 mm s⁻¹. The applied force for injection was measured. In one embodiment, the cement composition has an applied force for injection (Inj. force) ranging from about 2 to about 11 N or from about 6 to about 16 N; when measured by the above extrusion method. In one specific embodiment, the cement composition has an applied force for injection (Inj. force) ranging from about 2 to about 10 N, preferably ranging from about 3 to about 9 N, more preferably ranging from about 4 to about 8 N; when measured by the above extrusion method.

The setting time correspond to an early stage of the setting reaction, whereas the end of the setting reaction is usually reached after a few days. Advantageously, the setting time ranges from about 2 to about 30 min, preferably between about 2 to about 8 min or between about 8 to about 30 min. The setting time is a significant property regarding the use as bone substitute: if the setting time is too fast, there is not enough time to use the cement before it hardened. If the setting time is too long, it is necessary to wait before closing the wound.

Methods for measuring a setting time of a cement composition are well-known in the art. Gillmore needle method (ASTM C266-2018) and the Vicat needle method (ASTM C191-92) are based on the measure of static pressure and commonly used, although others techniques are known (X-ray diffraction analysis, infrared spectroscopy or measure of the pH of the cement paste), including non-destructive methods (thermal analysis, calorimetry, ultrasound-based methods or AC impedance spectroscopy).

According to one embodiment, the cement composition has a setting time (ST) ranging from about 2 to about 20 min; when measured by the Gillmore needle method (ASTM C266-2018). In one embodiment, the cement composition has a ST ranging from about 4 to about 7 min or from about 10 to about 16 min; when measured by the Gillmore needle method (ASTM C266-2018).

The setting time may be reduced when large concentrations of phosphate ions are present in the mixing solution, especially because the chemical reaction consuming phosphate is accelerated (by application of Le Chatelier principle). According to one embodiment, at least one water-soluble phosphate salt is added in the powder component. Upon contact with the liquid component, the phosphate salt dissolves into the liquid component.

According to one embodiment, at least one water-soluble phosphate salt is added in the liquid component. In one embodiment, the soluble phosphate salts are selected from ammonium dihydrogen phosphate (NH₄H₂PO₄), disodium hydrogen phosphate (Na₂HPO₄), monosodium dihydrogen phosphate (NaH₂PO₄), dipotassium hydrogen phosphate (K₂HPO₄), monopotassium dihydrogen phosphate (KH₂PO₄) and mixtures thereof.

Others compounds may be added in the liquid component in order to reduce the setting time. According to one embodiment, the liquid component comprises at least one compound selected from dilute organic acids (such as acetic acid, citric acid or succinic acid), sodium carbonate, sodium bicarbonate, sodium alginate, sodium citrate, sodium chondroitin sulphate and mixtures thereof.

Advantageously, the cement composition adheres sufficiently to bone tissue before and during the setting. Sufficient adhesion to bone tissue is advantageous regarding the use as bone substitute because, in many applications, the bone is bleeding and the blood jeopardizes the needed contact between the bone cement and bone tissue and then the aimed osteoconductive process. Methods for measuring adhesion are well-known in the art.

Advantageously, the cement composition is resistant to blood wash. Blood wash resistance is advantageous regarding the use as bone substitute because if the cement paste is not resistant enough, it will be decomposed before the setting process has occurred.

Advantageously, the cement composition is biocompatible, as defined hereinabove.

Bone Cement

This invention also relates to a bone cement obtainable by a process comprising the following steps:
(i) the preparation of a cement composition comprising hyaluronic acid (HA) as described hereinabove by mixing:
   a powder component as described hereinabove; and
   a liquid component as described hereinabove; and

(ii) the setting of the cement composition.

This invention also relates to a bone cement obtainable by setting a cement composition as described hereinabove.

According to one embodiment, the bone cement is a calcium phosphate cement (CPC).

The bone cement according to the invention is a porous material. Porous material may be characterized by porosity (total volume), but also by the size, shape and/or connectivity of pores. According to one embodiment, the bone cement is microporous. According to one embodiment, the bone cement is mesoporous. According to one embodiment, the bone cement is microporous. Methods for measuring parameters characterizing a porous material are well-known in the art. Porosity may for example be measured by intrusion of a liquid (e.g., mercury) or a gas (e.g., argon or nitrous oxide) in the cement. Size, shape and/or connectivity of pores may for example be measured by observation of the cement with a scanning electron microscope (SEM) or determined by gas adsorption. According to one embodiment, the cement has a lower mineral density, which results in a high porosity, i.e., has a highly open structure. Structures of high porosity are advantageous for bone cement applications in terms of cellular colonization and cement resorption.

Advantageously, the bone cement has mechanical properties appropriate for use as bone substitute, i.e. mechanical properties within the same order than the mechanical properties of the trabecular bone. Appropriate mechanical properties of the cement paste are advantageous regarding the use as bone substitute because the bone cement is generally in direct contact with the bone, so that similar mechanical properties improves the cooperation and durability of the bone repair. Parameters commonly measured to characterize the mechanical properties of cements includes Young's Modulus (YM), ultimate tensile strength (UTS), shortened to “tensile strength” (TS), and compressive strength (Rs). Methods for measuring Young's Modulus (YM), tensile strength and compressive strength are well-known in the art (e.g., as described in Kaplan et al., Journal of Biomechanics, 1985 and Zhang et al. Acta Biomaterialia, 2014).

Typically, bone cements have a tensile strength ranging from 1 to 10 MPa and a compressive strength ranging from 10 to 100 MPa, whereas the trabecular bone has a tensile strength ranging from 5 to 15 MPa and a compressive strength ranging from 2 to 25 MPa. According to one embodiment, the cement has a compressive strength (Rₛ) ranging from about 5 to about 25 MPa, preferably ranging from about 7 to about 20 MPa, more preferably ranging from about 9 to about 17 MPa. According to one embodiment, the cement has a Young's Modulus (YM) ranging from about 500 to about 950 MPa, preferably ranging from about 550 to about 900 MPa, more preferably ranging from about 600 to about 850 MPa.

Advantageously, the bone cement has a durability appropriate for use as bone substitute, i.e., has a high toughness. Toughness of a bone substitute cement is considered “low” if ranging from 0.010 to 0.050 kJ/m² in their work of fracture. Toughness of a bone substitute cement is considered high if ranging from 0.5 to 15 kJ/m² in their work of fracture. Sufficient resistance of the cement is advantageous regarding the use as bone substitute because a bone substitute should not break after it has been installed and is also indented to keep its physical integrity until it has achieved significant resorption.

Advantageously, the bone cement is biocompatible. Advantageously, the bone cement is bioresorbable.

Uses

This invention also relates to a cement composition as described hereinabove for use in the treatment of a dental defect or fracture or for use in the treatment of a bony defect or fracture. According to one embodiment, the dental or bony defect or fracture is caused by trauma and/or associated with osteoporosis.

This invention also relates to a method of treatment of a dental or bony defect or fracture in a subject in need thereof, comprising a step of administration of a cement composition as described hereinabove or of a bone cement as described hereinabove to a bone of the subject. This invention also relates to the use of a cement composition as described hereinabove or of a bone cement as described hereinabove, in the manufacture of a medicament for the treatment of a dental or bony defect or fracture.

This invention also relates to the use in vivo, in vitro or ex vivo of a cement composition as described hereinabove or of a bone cement as described hereinabove, in the manufacture of a dental or bony implant. In one embodiment, the use is in vitro or ex vivo.

This invention also relates to the use in vivo, in vitro or ex vivo of a bone cement as described hereinabove as a scaffold for tissue engineering. In one embodiment, the use is in vitro or ex vivo.

Implant

This invention also relates to a dental or bony implant consisting of a moulding of a bone cement as described hereinabove.

Kit-of-Parts

This invention also relates to kit-of-part for manufacturing a cement composition comprising hyaluronic acid (HA) as described hereinabove or a bone cement comprising hyaluronic acid (HA) as described hereinabove, comprising:
a first part being a powder component as described hereinabove; and
a second part being a liquid component as described hereinabove.

According to one embodiment, the bone cement is a calcium phosphate cement (CPC).

### BRIEF DESCRIPTION OF THE DRAWINGS

FIG. 1 is a series of photographs showing the aspect of cement compositions HBS, 3H and 31 after injection and after hardening, for qualitative evaluation of their cohesiveness as described in Example 3.

FIG. 2 is a series of photographs showing the aspect of cement compositions 3J, 4H and 5J after injection and after hardening, for qualitative evaluation of their cohesiveness as described in Example 3.

FIG. 3 is a series of photographs showing the microstructure characterization (SEM) of cement HBS, 3H and 3J as described in Example 3. The structure of the cements 3H and 3J have a lower mineral density than HBS cement, so that the cements according to the invention results have a higher porosity.

FIG. 4 is a series of photographs showing the aspect of cement compositions 3H and M after injection and after hardening, for qualitative evaluation of their cohesiveness as described in Example 4.

### EXAMPLES

The present invention is further illustrated by the following examples.

### Example 1

### Bone Cement Compositions and Manufacture thereof

Materials and Methods

Materials

Sodium hyaluronate (NaHA) with molecular weight of about 800 kDa, hydroxypropylmethylcellulose (HPMC) and inorganic salts were purchased from commercial providers and used without further purification.

Methods

Liquid component preparation: Solid sodium hyaluronate (NaHA) was dissolved in a disodium hydrogen phosphate (Na₂HPO₄) aqueous solution so as to obtain the liquid component.

Powder component preparation: The solid substances were mechanically mixed together so as to obtain the powder component. The powder component has a main population of particle size (i.e., more than 50% of particles in the powder) ranging from 3 to 30 μm.

Bone cement preparation: The powder component was placed in a mortal and the liquid component was added thereto, then the mixture was mixed at room temperature with a metal spatula until a homogeneous paste was obtained (about 1 min). Reference time to is defined as the moment of the liquid-powder contact.

Results

The disclosed bone cement compositions have the following compositions:

TABLE 1

| | Composition | | | |
|---|---|---|---|---|
| Powder component (% w/w) | A | B | | C |
| α-tricalcium phosphate | 67.5 ± 2.5% | 77.5 ± 2.5% | | 87.5 ± 2.5% |
| (α-TCP) | | | | |
| dicalcium phosphate | 14.8 ± 0.3% | 9.8 ± 0.3% | | 4.8 ± 0.3% |
| anhydrous (DCPA) | | | | |
| calcium-deficient | 15.1 ± 0.3% | 10.1 ± 0.3% | | 5.1 ± 0.3% |
| hydroxyapatite (CDA) | | | | |
| hydroxypropylmethylcellulose | 2.9 ± 0.1% | 1.9 ± 0.1% | | 0.9 ± 0.1% |
| (HPMC) | | | | |
| Liquid component (% w/w) | A | B | | C |
| disodium hydrogen phosphate | 0.50 ± 0.02% | 0.50 ± 0.02% | | 0.50 ± 0.02% |
| (Na₂HPO₄) | | | | |
| water | q.s. | q.s. | | q.s. |
| Liquid/powder ratio (L/S) | 0.460 ± 0.015 | 0.460 ± 0.015 | | 0.460 ± 0.015 |
| (mL/g) | | | | |
| | Composition | | | |
| Powder component (% w/w) | D | E | F | G |
| α-TCP | 77.5 ± 2.5% | 77.5 ± 2.5% | 77.5 ± 2.5% | 77.5 ± 2.5% |
| DCPA | 9.8 ± 0.3% | 9.8 ± 0.3% | 9.8 ± 0.3% | — |
| CDA | 10.1 ± 0.3% | 10.1 ± 0.3% | 10.1 ± 0.3% | 10.1 ± 0.3% |
| dicalcium phosphate | — | — | — | 5.2 ± 0.2% |
| dihydrate (DCPD) | | | | |
| monocalcium | — | — | — | 4.9 ± 0.2% |
| phosphate | | | | |
| monohydrate | | | | |
| (MCPM) | | | | |
| Liquid component (% w/w) | D | E | F | G |
| Na₂HPO₄ | 0.50 ± 0.02% | 0.50 ± 0.02% | 0.50 ± 0.02% | 5.0 ± 0.3% |
| water | q.s. | q.s. | q.s. | q.s. |
| L/S (mL/g) | 0.460 ± 0.015 | 0.510 ± 0.015 | 0.560 ± 0.015 | 0.510 ± 0.015 |

Sodium hyaluronate (NaHA) is further present in the liquid component in the following amounts:

TABLE 2

| | 1 | 2 | 3 | 4 |
|---|---|---|---|---|
| NaHA (% w/w) | — | 0.1% | 0.5% | 1% |

The cement compositions have been successfully manufactured using the materials and methods as described hereinabove.

Composition 1 is the control cement without hyaluronic acid. Compositions 2-4 are compositions of the invention.

### Example 2

### Properties of the Compositions

Materials and Methods

Materials

The bone cement compositions were manufactured as described in Example 1 above.

Methods

Measurement of Injectability—Extrusion Method: After Mixing the Solid and Liquid components, the cement composition (2.5 mL) was introduced in a syringe (Terumo®) with a 1.5 mm diameter needle, then was extruded by applying a syringe plunger displacement at a speed of 15 mm min−1.

Measurement of setting time—Gillmore needle method (ASTM C266-2018): The principle of this method is visual examination of the surface of the cement composition, the setting time being the moment at which a needle (with a given diameter and a weight) leaves only a small footprint when it is placed on the surface of the cement composition. Gillmore needle system consists of two needles: the first needle has a large diameter and a small weight and it is used to measure the initial setting time, noted t, whereas the second needle has a smaller diameter and a larger weight and it is used to measure the final setting time, noted t_{f}. The clinical meaning of t, and t_{f} is that the cement can be implanted until t, and that the wound can be closed as from t_{f}. Between tᵢ and t_{f}, the cement composition must not be touched nor deformed, as the hardening process is ongoing.

Measurement of Young's Modulus (YM) [as described in Kaplan et al., J. Biomech, 1985 and Zhang et al. Acta Biomater. 2014] and compressive strength (Rₛ): After mixing the solid and liquid components, the cement composition was moulded before being placed in a NaCl aqueous solution at 37° C. (similar to in vivo conditions). The measurement of diametric tensile strength and compressive strength were carried out at different times in order to follow the evolution of the mechanical properties during the setting of the cement until the setting reaction is considered complete (generally from 24 to 72 hours).

Results

Injected volume (Inj.), applied force for injection (Inj, force), setting time (ST), Young's Modulus (YM) and compressive strength (Rₛ) of compositions 1-4B are shown on

Table 3:

TABLE 3

| | Inj. | Inj, force | ST | YM | R_{S} |
|---|---|---|---|---|---|
| # | (mL) | (N) | (min) | (MPa) | (MPa) |
| 1B (0%) | 2.5 | 5.23 ± 2.31 | 8.0 | 891 ± 47 | 23.1 ± 1.3 |
| 2B (0.1%) | 2.5 | 5.88 ± 1.22 | 7.5 | 802 ± 66 | 22.5 ± 0.8 |
| 3B (0.5%) | 2.5 | 5.12 ± 2.77 | 7.0 | 701 ± 90 | 21.6 ± 1.5 |
| 4B (1%) | 2.5 | 8.11 ± 1.87 | 5.0 | 569 ± 54 | 18.1 ± 0.9 |

Applied force for injection tends to be similar—and within adequate working range—for all tested compositions 1-4B, although a limited increase is observed when high NaHA amounts are used (4B).

Gillmore setting time is significantly reduced in presence of NaHA in compositions 2-4B, depending of the amount of NaHA (from −6% to −38%).

Young's modulus is also significantly reduced in presence of NaHA in compositions 2-4B, depending of the amount of NaHA (from −10% to −36%). This reduced rigidity is interesting for bone filling applications.

Compressive strength tends to be similar—and within adequate working range—for all tested compositions 1-4B.

Therefore, the above results evidence that addition of hyaluronic acid or a salt thereof to the liquid component of a bone cement composition unexpectedly lead to significant improvement of physical and mechanical properties for both the cement composition (paste) and the bone cement (final material). Increasing amounts of HA ranging from 0.1 to 1% w/w in liquid component lead to increase of the properties relatively to the HA amount.

In order to study the effect of L/S ratio on the properties of the compositions, injectability (Inj.) and setting time (ST) of compositions 1D (control) and 3E-F (0.5% w/w NaHA, increasing L/S ratio from 0.45 to 0.55 mL/g) have been measured. The results are shown on Table 4:

TABLE 4

| | Inj. (Force (N), | ST |
|---|---|---|
| # | % extrusion) | (min) |
| ID | >25N | 3.7 ± 0.3 min |
| | 41% | |
| 3E | 59.4 ± 5.2N | 7.0 ± 0.5 min |
| | 94% ± 1% | |
| 3F | 28.7 ± 4.7N | 7.7 ± 0.3 min |
| | 94% ± 3% | |

In the compositions of the invention 3E and 3F, injectability (filter pressing, 4 min, 4 measures) increases as L/S ratio increases, whereas applied force decreases.

As L/S ratio increases, setting time (ST) (3 measures) also increases in compositions 3E and 3F compared to control 1D, but however remains within adequate working range (7-8 min).

Therefore, increasing the L/S ratio in the bone cement compositions of the invention may be an interesting option when increase of injectability is desired.

### Example 3

### Comparative Experiments over Graftys® HBS

The properties of the compositions according to the invention were compared with the properties of Graftys® HBS bone cement composition.

Materials and Methods

Materials

Graftys® HBS bone cement composition (“HBS” composition in Table 5 below), sodium hyaluronate (NaHA) with molecular weight of about 800 kDa and inorganic salts were purchased from commercial providers and used without further purification.

Methods

Manufacturing methods: Liquid component preparation: NaHA was dissolved in a disodium hydrogen phosphate (Na₂HPO₄) aqueous solution to obtain the liquid component. Powder component preparation: The solid substances were mechanically mixed together to obtain the powder component. The powder component has a main population of particle size (i.e., more than 50% of particles in the powder) ranging from 2 to 100 μm. Cement preparation: The powder component was placed in a mortar and the liquid component was added to it, then the mixture was mixed at room temperature with a pestle until a homogeneous paste was obtained (about 1 min). Reference time to is defined as the moment of the liquid-powder contact.

Characterization methods: Measurement of injectability & cohesiveness—Extrusion method: After mixing the solid and liquid components, the cement composition (3.5 mL) was introduced in a syringe (Terumo®) with a 1.5 mm diameter needle. After 15 min, the paste was extruded by applying a syringe plunger displacement at a speed of 1 mm s⁻¹.

The applied force for injection was measured. Cohesiveness was qualitatively evaluated by taken a picture just after injection in a physiological solution and after 24 h or 72 h of incubation at 37° C. in the same solution. Measurement of initial setting time—Gillmore needle method (ASTM C266-2018): The principle of this method is visual examination of the surface of the cement composition, the setting time being the moment at which a needle (with a given diameter and a weight) leaves only a small footprint when it is placed on the surface of the cement composition. Measurement of compressive strength (Rₛ) and Young's Modulus (YM): After mixing the solid and liquid components, cement paste was molded in cylinders before being placed in a physiological solution at 37° C. (similar to in vivo conditions). After 72 h, at least 5 porous cylinders per condition were submitted to increasing compression load (compressive displacement of 1 mm s⁻¹). From the obtained stress vs. strain curve, compressive strength of the material was evaluated. Young's Modulus (YM) was evaluated as the slope of the stress-strain curve in the elastic region (linear region) [as described in Kaplan et al., J. Biomech, 1985 and Zhang et al. Acta Biomater. 2014]. Microstructure characterization by SEM: The measurements were carried out on cylindrical cement blocks allowed to harden for 72h in 0.9 wt. % NaCl at 37° C. Then, a 1 mm² polished cross-section of the samples was obtained using a JEOL cross section polisher SM09010, by applying an argon ion beam accelerated by a voltage from 4.5 to 6 kV perpendicular to the surface of each specimen for 4 to 8 hours. SEM observation of those samples was performed using a Field Emission Gun Scanning

Electron Microscope (Jeol 7600F). Images were acquired on a back scattered electron mode with an 8 pA beam current and a 8 kV accelerated voltage.

Results

The compared bone cements have the following compositions:

TABLE 5

| | Composition | | | |
|---|---|---|---|---|
| Powder component (% w/w) | HBS | H | 1 | J |
| α-tricalcium phosphate | 77.5 ± 2.5% | 79.5 ± 2.5% | 79.5 ± 2.5% | 79.5 ± 2.5% |
| (α-TCP) | | | | |
| dicalcium phosphate | 5.2 ± 0.2% | 5.1 ± 0.2% | 5.1 ± 0.2% | 5.1 ± 0.2% |
| dihydrate (DCPD) | | | | |
| monocalcium phosphate | 4.9 ± 0.2% | 5.2 ± 0.2% | 5.2 ± 0.2% | 5.2 ± 0.2% |
| monohydrate (MCPM) | | | | |
| calcium-deficient | 10.1 ± 0.3% | 10.1 ± 0.3% | 10.1 ± 0.3% | 10.1 ± 0.3% |
| hydroxyapatite (CDA) | | | | |
| Hydroxy-propyl-methylcellulose | 1.9 ± 0.1% | — | — | — |
| (HPMC) | | | | |
| Liquid component (% w/w) | HBS | H | 1 | J |
| disodium hydrogen | 5.1 ± 0.2% | 5.1 ± 0.2% | 5.1 ± 0.2% | 5.1 ± 0.2% |
| phosphate (Na₂HPO₄) | | | | |
| water | q.s. | q.s. | q.s. | q.s. |
| Liquid/powder ratio (L/S) (mL/g) | 0.59 ± 0.015 | 0.59 ± 0.015 | 0.50 ± 0.015 | 0.65 ± 0.015 |

Sodium hyaluronate (NaHA) is further present in the liquid component in the following amounts:

TABLE 6

| | 3 | 4 | 5 |
|---|---|---|---|
| NaHA (% w/w) | 0.5 | 1 | 0.75 |

Composition HBS is the control cement. HBS comprises HPMC and does not comprise hyaluronic acid. Graftys® HBS is a state-of-the art commercial bone cement composition.

The interest of adding HPMC as organic component in the powder of Graftys® HBS is disclosed in WO 2008/023254 Al patent application (Khairoun, I. et al.). Compositions 3H, 4H, 3I, 3J and 5J are compositions according to the invention.

Measured applied force for injection (Inj, force), setting time (ST), compressive strength (Rₛ) and Young's Modulus (YM) of the cement compositions are shown on

Table 7:

TABLE 7

| | Inj, force | ST | Rₛ | YM |
|---|---|---|---|---|
| # | (N) | (min) | (MPa) | (MPa) |
| HBS | 12.67 ± 2.68 | 12.75 ± 0.25 | 8.43 ± 1.96 | 367 ± 81 |
| 3H | 7.50 ± 1.43 | 11.06 ± 0.25 | 13.28 ± 0.85 | 836 ± 35 |
| 3I | 6.16 ± 0.30 | 11.46 ± 0.39 | 15.63 ± 2.31 | 765 ± 92 |
| 3J | 3.00 ± 1.10 | 14.64 ± 0.32 | 9.92 ± 1.33 | 671 ± 72 |
| 4H | 9.50 ± 1.38 | 12.13 ± 0.18 | 14.51 ± 1.29 | 824 ± 37 |
| 5J | 3.85 ± 0.54 | 13.67 ± 0.28 | 9.93 ± 2.08 | 598 ± 108 |

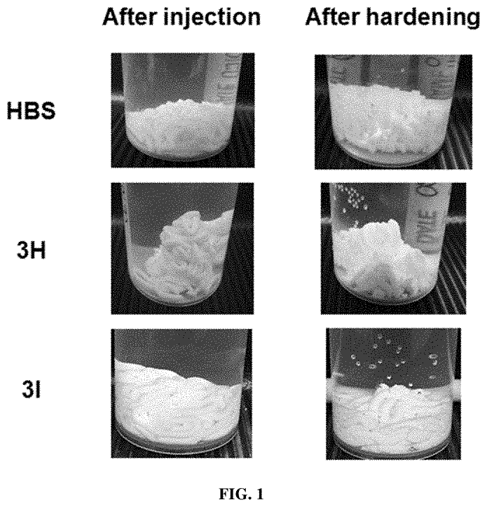

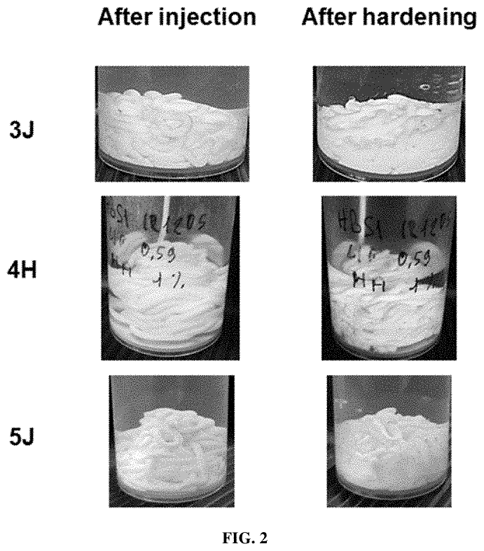

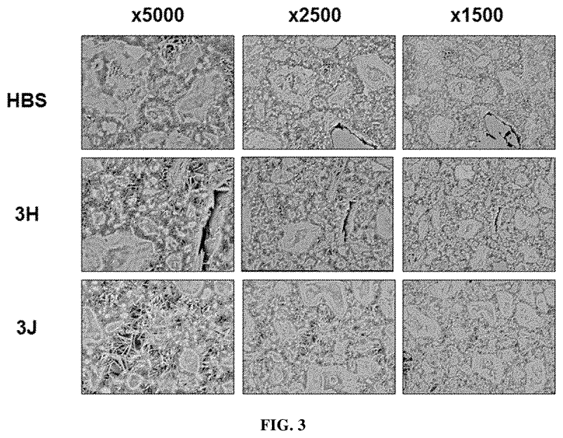

FIG. 1 and FIG. 2 show the cohesiveness of the cement compositions after injection and after hardening. FIG. 3 shows the microstructure comparison between HBS and 3H and 3J.

Injectability is significantly increased in the compositions according to the invention, as evidenced by the applied force for injection that is significantly reduced when the liquid component comprises NaHA (up to −76%).

Increasing the L/S ratio up to 0.65 significantly increases injectability in 3J, whereas the difference in L/S ratio between 3H and 3I (0.59 and 0.50 respectively) does not significantly impact the decrease in the force for injection. At L/S constant ratio, a significant increase in hyaluronic acid concentration come up with an increase in the viscosity of the liquid phase and therefore, the applied force for injection is increased in 4H in comparison with 3H. By contrast, a small difference in NaHA concentration (from 0.5 to 0.75 wt. %) does not significant affect the applied force for injection in 3J and 5J.

In any case, the injectability of all the compositions according to the invention (3H, 3I, 3J, 4H and 5J) is significantly improved compared to prior art composition (HBS).

Moreover, the cohesiveness is increased in the compositions according to the invention (3H, 3I, 3J, 4H and 5J) comprising NaHA compared to prior art composition (HBS) (FIG. 3): a higher shape retention of the cement filaments after injection into an aqueous solution is observed in 3H, 3I, 3J, 4H and 5J than in HBS. Especially, no disintegration is observed for 3H, 3I, 3J, 4H and 5J after injection.

Higher injectability and cohesiveness are very interesting properties for bone filling applications.

Gillmore setting time (ST) tends to be similar for all tested compositions.

Compressive strength (Rs) is increased (from +58% to +85%) in the cements 3H, 3I and 4H according to the invention, which have the same or lower L/S ratio as HBS.

Young's modulus is increased (up to +128%) in all the cements according to the invention (3H, 3I, 3J, 4H and 5J) compared to prior art composition (HBS).

In the microstructure analysis, different types of particles are observed for HBS and the cements according to the invention (3H and 3J). The size distribution ranges from 1.4 to 32 μm and the average size is 9 μμm. These particles correspond to (i) a dense small amount, either along their whole cross-section or in their inner part, and correspond to unreacted or partially hydrolyzed α-TCP or DCPD; (ii) a large amount of particles that have a geode-like morphology with a dense shell lined in its inner part with interdigitated platelet crystals, as a result of a full hydrolysis of α-TCP into CDA. The intergranular space (i.e., the area in between all these particles) is mostly occupied by CDA platelet crystals forming channels with a “sand rose” architecture. From FIG. 3 it is evidenced that the replacement of HPMC in the cement powder with NaHA in the liquid phase leads to a lower mineral density and higher particle size in the intergranular space.

As a consequence, the cements 3H and 3J according to the invention have a more open structure, of high porosity and with mechanical properties similar or superior to HBS, with less polymer added in the cement (about 85% less polymer in the cement). The latest characteristics would have a positive impact on cell colonization and the resorption of cement.

Therefore, the above results clearly evidence that the addition of hyaluronic acid or a salt thereof to the liquid component instead of HPMC in the powder component of a bone cement composition lead to significant improvement of physical and mechanical properties for both the cement paste and the bone cement (final material).

### Example 4

### Effect of Hyaluronic Acid in Liquid or Solid Component

The effect of adding hyaluronic acid either in the liquid component or in the solid component has been studied.

Materials and Methods

Materials

Sodium hyaluronate (NaHA) with molecular weight of about 800 kDa and inorganic salts were purchased from commercial providers and used without further purification.

Methods

Manufacturing methods: Liquid component preparation: NaHA was dissolved in a disodium hydrogen phosphate (Na₂HPO₄) aqueous solution to obtain the liquid component. Cement powder preparation: The solid substances were mechanically mixed together to obtain the powder component. NaHA-cement powder preparation: NaHA filaments were mixed with the cement powder overnight so as to obtain the powder component. The powder component has a main population of particle size (i. e., more than 50% of particles in the powder) ranging from 2 to 100 μm. Cement preparation: The powder component was placed in a mortar and the liquid component was added to it, then the mixture was mixed at room temperature with a pestle until a homogeneous paste was obtained (about 1 min). Reference time to is defined as the moment of the liquid-powder contact.

Characterization methods: Measurement of injectability & cohesiveness—Extrusion method: After mixing the solid and liquid components, the cement composition (3.5 mL) was introduced in a syringe (Terumo®) with a 1.5 mm diameter needle. After 15 min, the paste was extruded by applying a syringe plunger displacement at a speed of 1 mm s-1.

The applied force for injection was measured. Cohesiveness was qualitatively evaluated by taken a picture just after injection in a physiological solution and after 24h or 72h of incubation at 37° C. in the same solution. Measurement of initial setting time—Gillmore needle method (ASTM C266-2018): The principle of this method is visual examination of the surface of the cement composition, the setting time being the moment at which a needle (with a given diameter and a weight) leaves only a small footprint when it is placed on the surface of the cement composition.

Results

The compared bone cements have the following compositions:

TABLE 8

| | Composition | |
|---|---|---|
| Powder component (% w/w) | 3H | M |
| α-tricalcium phosphate | 79.5 ± 2.5% | 79.5 ± 2.5% |
| (α-TCP) | | |
| dicalcium phosphate | 5.1 ± 0.2% | 5.1 ± 0.2% |
| dihydrate (DCPD) | | |
| monocalcium phosphate | 5.2 ± 0.2% | 5.2 ± 0.2% |
| monohydrate (MCPM) | | |
| calcium-deficient | 10.1 ± 0.3% | 10.1 ± 0.3% |
| hydroxyapatite (CDA) | | |
| Sodium hyaluronate | — | 0.28 ± 0.02% |
| (NaHA) | | |
| Liquid component (% w/w) | 3H | M |
| Sodium hyaluronate | 0.50 ± 0.02% | — |
| (NaHA) | | |
| disodium hydrogen | 5.1 ± 0.2% | 5.1 ± 0.2% |
| phosphate (Na₂HPO₄) | | |
| water | q.s. | q.s. |
| Liquid/powder ratio | 0.59 ± 0.015 | 0.59 ± 0.015 |
| (L/S) (mL/g) | | |

10

Compositions 3H and M are compositions according to the invention. 3H has the sodium hyaluronate in the liquid component, whereas M has the sodium hyaluronate in the powder component. This is the only difference between compositions 3H and M because, due to the L/S ratio of 0.59, the concentration of sodium hyaluronate after mixing the liquid and solid component is actually the same (about 0.2% w/w).

Measured applied force for injection (Inj, force) and setting time (ST) of the cement compositions are shown on Table 9:

TABLE 9

| | Inj, force | ST |
|---|---|---|
| # | (N) | (min) |
| 3H | 7.50 ± 1.43 | 11.06 ± 0.25 |
| M | 7.02 ± 1.72 | 12.88 ± 0.25 |

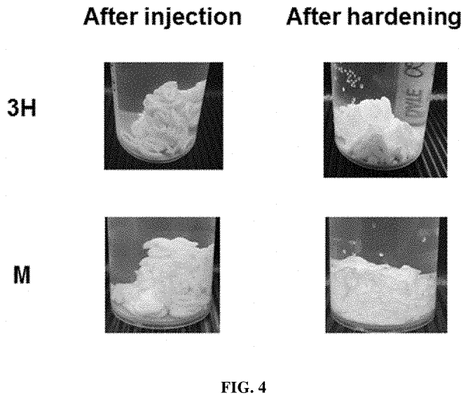

FIG. 4 shows the cohesiveness of the cement compositions after injection and after hardening.

The applied force for injection is low in presence of NaHA, whether it is in the liquid component (3H) or in the powder component (M). Additionally, cohesiveness is high and adequate (FIG. 4). The low applied forces for injection and the cohesiveness make those compositions fully injectable and easy-to-use, and thus of high interest for bone filling applications.

Gillmore setting time is in the adequate range of low viscosity bone cement paste for both compositions according to the invention (3H and M).

Therefore, the above results unambiguously evidence that the addition of hyaluronic acid or a salt thereof in a bone cement composition leads to similar properties, whether it is added in the liquid component and/or in the powder component.

1-21. (canceled)

22. A bone cement composition comprising:
a powder component comprising:
calcium phosphate compounds comprising at least alpha-tricalcium phosphate (α-TCP) and comprising at least calcium-deficient apatite (CDA), and
optionally at least one calcium sulfate compound;
a liquid component; and
hyaluronic acid (HA) or a salt thereof;
wherein:
said hyaluronic acid (HA) or salt thereof is comprised in said powder component in an amount ranging from 0.01% to 10% w/w;
and/or
said hyaluronic acid (HA) or salt thereof is comprised in said liquid component in an amount ranging from 0.01% to 10% w/w.

23. The bone cement composition according to claim 22, wherein said liquid component is an aqueous solution or an aqueous suspension.

24. The bone cement composition according to claim 22, wherein said powder component comprises said α-TCP in an amount ranging from 60% to 95% w/w.

25. The bone cement composition according to claim 22, wherein said powder component comprises said CDA in an amount ranging from 2.5% to 20% w/w.

26. The bone cement composition according to claim 22, wherein said powder component comprises at least one further calcium phosphate compound selected from the group consisting of hydroxyapatite (HAₚ), amorphous calcium phosphate (ACP), monocalcium phosphate anhydrous (MCPA), monocalcium phosphate monohydrate (MCPM), dicalcium phosphate dihydrate (DCPD), dicalcium phosphate anhydrous (DCPA), β-tricalcium phosphate (β-TCP), octacalcium phosphate (OCP), tetracalcium phosphate (TTCP), and a mixture thereof; and/or at least one calcium sulfate compound selected from the group consisting of anhydrous calcium sulfate, calcium sulfate hemihydrate, calcium sulfate dihydrate, and a mixture thereof.

27. The bone cement composition according to claim 22, wherein said powder component consists of α-TCP, CDA and DCPA or consists of α-TCP, CDA, DCPD and MCPM.

28. The bone cement composition according to claim 22, wherein said liquid component further comprises at least one inorganic salt selected from the group consisting of sodium chloride (NaCl), trisodium phosphate (Na₃PO₄), disodium hydrogen phosphate (Na₂HPO₄), monosodium dihydrogen phosphate (NaH₂PO₄), and a mixture thereof; wherein said inorganic salt is in an amount ranging from 0.01% to 20% w/w.

29. The bone cement composition according to claim 22, wherein said liquid component further comprises at least one inorganic salt selected from the group consisting of Na₂HPO₄ and a Na₂HPO_{4/}NaH₂PO₄ mixture; wherein said inorganic salt is in an amount ranging from 0.1% to 1% w/w or from 2 to 8% w/w.

30. The bone cement composition according to claim 22, wherein said liquid component comprises at least one biological liquid selected from the group consisting of blood, plasma, platelets, platelets concentrate, bone marrow, bone marrow concentrate, and mixtures thereof.

31. The bone cement composition according to claim 22, wherein said liquid component consists of water, HA or salt thereof, optionally Na₂HPO₄, and optionally at least one biological liquid selected from the group consisting of blood, plasma, platelets, platelets concentrate, bone marrow, bone marrow concentrate, and mixtures thereof.

32. The bone cement composition according to claim 22, wherein said HA or salt thereof has a molecular weight ranging from 1 kDa to 5 000 kDa.

33. The bone cement composition according to claim 22, wherein said powder component comprises said HA or salt thereof in an amount ranging from 0.01% to 5% w/w.

34. The bone cement composition according to claim 33, wherein said powder component comprises said HA or salt thereof in an amount ranging from 0.1% to 1% w/w.

35. The bone cement composition according to claim 22, wherein said liquid component comprises said HA or salt thereof in an amount ranging from 0.01% to 5% w/w.

36. The bone cement composition according to claim 35, wherein said liquid component comprises said HA or salt thereof in an amount ranging from 0.1% to 1% w/w.

37. The bone cement composition according to claim 22, wherein the liquid component/powder component (L/S) ratio ranges from 0.3 to 0.7 ml/g.

38. The bone cement composition according to claim 22, wherein said bone cement composition is injectable.

39. The bone cement composition according to claim 22, further comprising at least one active ingredient selected from the group consisting of antibiotics, anti-inflammatory drugs, anti-cancer drugs, anti-osteoporosis drugs, bone anabolic drugs, growth factors, water-soluble radiopaque agents, contrast agents, and a mixture thereof.

40. A bone cement obtainable by a process comprising the following steps:
the preparation of a bone cement composition according to claim 22, and the setting of said bone cement composition.

41. A kit-of-parts for manufacturing a bone cement composition according to claim 22, said kit-of-parts comprising:
a first part being a powder component comprising:
calcium phosphate compounds comprising at least alpha-tricalcium phosphate (α-TCP) and comprising at least calcium-deficient apatite (CDA), and optionally at least one calcium sulfate compound;
a second part being a liquid component; and
hyaluronic acid (HA) or a salt thereof;
wherein:
said hyaluronic acid (HA) or salt thereof is comprised in said powder component in an amount ranging from 0.01% to 10% w/w;
and/or
said hyaluronic acid (HA) or salt thereof is comprised in said liquid component in an amount ranging from 0.01% to 10% w/w.
